(19) United States
(12) Patent Application Publication
(10) Pub. No.: US 2010/0172875 A1
(43) Pub. Date: Jul. 8, 2010

(54) OIL-IN-WATER EMULSION AND ITS USE FOR THE DELAYED RELEASE OF ACTIVE ELEMENTS

(75) Inventors: Van Anh Phan, Epalinges (CH); Nicolas Godinot, Lausanne (CH); Laurent Sagalowicz, Cully (CH); Martin Leser, Bretigny (CH); Fabien Robert, Divonne Les Bains (FR)

Correspondence Address: K&L Gates LLP, P.O. Box 1135, CHICAGO, IL, 60690, US

(73) Assignee: NESTEC S.A., Vevey (CH)

(21) Appl. No.: 12/602,339

(22) PCT Filed: May 30, 2008
(86) PCT No.: PCT/EP08/56717

§ 371 Date: Nov. 30, 2009

(30) Foreign Application Priority Data
May 30, 2007 (EP) PCT/EP07/055240

(51) Int. Cl. A61K 35/74; A61K 38/02; A61K 8/64; A61K 31/70; A61K 8/60; A61K 31/715; A61K 8/73; A61K 8/99; A61K 38/43; A61K 8/66; A61K 31/047; A61K 8/34; A61K 31/355; A61K 8/67; A61K 31/122; A61K 8/35; A61K 36/55; A61K 8/97; A61K 36/534; A61K 35/60; A61K 8/98; A61K 31/202; A61K 8/36; A61K 31/375; A61K 31/714; A61K 31/522; A61K 8/49; A61P 3/02; A61P 39/06; A23L 1/22; A61K 8/04

(52) U.S. Cl. 424/93.4; 514/2; 514/23; 514/54; 424/94.1; 514/729; 514/458; 514/690; 424/768; 424/747; 424/523; 514/560; 514/474; 514/52; 514/263.34; 512/2; 426/650

(57) ABSTRACT

The present invention concerns the use of an oil-in-water emulsion where the interior of oil droplets exhibit interfaces, between lipophilic domains and hydrophilic or amphiphilic domains, due to the presence of a lipophilic additive solubilized inside the oil droplets and which is used for delayed release of active elements such that the release of at least one active element, which has a octanol/water partitioning coefficient logP higher than −1, corresponds to a higher Tmax than the Tmax obtained for the simple reference oil-in-water emulsion where no lipophilic additive is used.

### FIELD OF INVENTION

The present invention concerns an oil-in-water emulsion that is used to delay the release of active elements.

### BACKGROUND OF THE INVENTION

### Emulsions in Industry

One of the uses of emulsions in Industry is to deliver active elements, such as, flavours, aromas vitamins, antioxidants, neutraceuticals, phytochemicals, drugs, chemicals, etc. Controlled release of the active elements requires the use of an appropriate vehicle for obtaining the desired release profile. Oil-in-water emulsions are commonly used delivery systems since they take advantage of the increased solubility of lipophilic active compounds in the oil. These kinds of emulsions are obtained using common lab-scale or industrial homogenizers.

If the oil droplets in the oil-in-water emulsions are ultra small, e.g. in the order of several nanometres to about 200 nm diameter, the emulsion is called oil-in-water microemulsion (Evans, D. F.; Wennerström, H. (Eds.); ‘The Colloidal Domain’, Wiley-VCH, New York, (1999)). These emulsions are clear and thermodynamically stable and, therefore, are for the man skilled in the art different from ordinary emulsions the latter being thermodynamically unstable and generally turbid. WO 2005/110370 described a new type of oil in water emulsion where the oil droplets are structured using a lipophilic additive (LPA) and where the oil droplet contains hydrophilic domain having size in the range 0.5 nm to 200 nm.

### Emulsions for the Delayed Release of Active Elements

As state of the art, dispersed oil droplets in products are used as delivery systems for molecules, such as aromas and nutrients which are dissolved in the oil droplets. One of the drawback of this kind of dispersions or emulsions as a vehicle system is that a high level of fat is required in order to have a delayed release of all the molecules and in particular of the more lipophilic ones (Bennett, C. J., “Formulating low-fat foods with good taste,” Cereal Foods World, Vol. 37, 1992, pp. 429-432). In particular, in vitro instrumental analysis and in vivo measurements reported an increase in the headspace and nose-space concentrations of lipophilic aroma compounds while lowering fat content in the media (Carey, M. E., Asquith, T., Linforth, R. S., and Taylor, A. J., “Modeling the partition of volatile aroma compounds from a cloud emulsion,” Vol. 50, No. 7, 2002, pp. 1985-1990; Miettinen, S. M., Tuorila, H., Piironen, V., Vehkalahti, K., and Hyvonen, L., “Effect of emulsion characteristics on the release of aroma as detected by sensory evaluation, static headspace gas chromatography, and electronic nose,” J. Agric. Food Chem., Vol. 50, No. 15, 2002, pp. 4232-4239 and Brauss, M. S., Linforth, R. S. T., Cayeux, I., Harvey, B., and Taylor, A. J., “Altering the fat content affects flavor release in a model yoghurt system,” Journal of Agricultural and Food Chemistry, Vol. 47, No. 5, 1999, pp. 2055-2059). The influence of fat content on the behaviour of lipophilic aroma compounds is well explained by the fact that fat acts naturally as solvent for lipophilic aroma compounds. When fat is reduced, these compounds are less retained by the matrix (Hatchwell, L. C., “Implications of fat on flavor,” Flavour-Food Interactions Washington, D.C., 1994, pp. 14-23) and consequently are more released into the water phase or into the air phase within or surrounding the matrix. For volatile compounds such as aromas the increased release in the water phase will drive an increase of release into the air phase within or surrounding the matrix.

### DESCRIPTION OF THE INVENTION

The present invention is based on the finding that the presence in the interior of oil droplets of interfaces, between lipophilic domains and hydrophilic or amphiphilic domains, created by the presence of a lipophilic additive solubilized in the interior of ordinary oil droplets or oil can lead to a delayed release of active elements from the oil-in-water emulsion of this invention. In particular this structure can lead to a delayed release of molecules present and in particular of the more lipophilic one and the delayed release effect can be obtained while maintaining a relatively low fat or oil level. The structures inside the oil droplets are formed by the addition of a lipophilic additive (denoted as LPA) to the oil droplets.

Example of active elements are flavors, flavor precursors, aromas, aroma precursors, taste enhancers, salts, sugars, amino-acids, polysaccharides, enzymes, peptides, proteins or carbohydrates, food supplements, food additives, hormones, bacteria, plant extracts, medicaments, drugs, nutrients, chemicals for agro-chemical or cosmetical applications, carotenoids, vitamins, antioxydants or nutraceuticals selected from the group comprising of lutein, lutein esters, β-carotene, tocopherol, tocopherol acetate, tocotrienol, lycopene, Co-Q₁₀, flax seed oil, fish oil, omega-3 oils, omega-6 oils, DHA, EPA, arachidonic-rich oils, LCPUFA oils, menthol, mint oil, lipoic acid, vitamins, polyphenols and their glycosides, ester and/or sulfate conjugates, isoflavones, flavonols, flavanones and their glycosides such as hesperidin, flavan 3-ols comprising catechin monomers and their gallate esters such as epigallocatechin gallate and their procyanidin oligomers, vitamin C, vitamin C palmitate, vitamin A, vitamin B₁₂, vitamin D, α- and/or γ-polyunsaturated fatty acids, phytosterols, esterified phytosterols, free, non esterified phytosterols, zeaxanthine, caffeine, and a combination thereof.

WO880059 describes a controlled release delivery system of active elements dissolved in a L2-phase. The structure of the L2 phase has some similarities with the structure of the oil droplet of the present invention. However in WO880059 the L2 structure was not dispersed into water and therefore could be considered as a pure oily system and did not form an oil-in-water emulsion. For the man skilled in the art, it cannot be forecast that if a controlled release system is obtained for a given structure, the same will be true when the controlled release system is dispersed in an aqueous system. For example, Boyd et al. (2003, Characterisation of drug release from cubosomes using the pressure ultrafiltration method, International Journal of Pharmaceutics, 260, 239-247) studied a similar system as the one described in the present invention. The non dispersed lipidic system corresponded to a delayed release of the dissolved active elements. However when the lipidic system was dispersed into water, no delayed release was observed and all the release of all active elements solubilized into the dispersed structure corresponded to a burst release.

U.S. Pat. No. 6,703,062 B1 describes a controlled released system based on oil encapsulation within gel particles. The oil droplets in the oil-in-water emulsions so formed are incorporated into gelled beadlets. The rationale is to create barriers around the oil droplets, which hinder the movement of the lipophilic aroma compounds from the oil phase into the aqueous continuous phase. The structure described in U.S. Pat. No. 6,703,062 B1 is very different from the oil-in-water emulsion of the present invention since, in the present invention the oil droplets are structured with the LPA and contain hydrophilic or amphiphilic domains which is not the case in U.S. Pat. No. 6,703,062B1.

### PRECISE DESCRIPTION OF THIS INVENTION

The present invention concerns the use of an oil-in-water emulsion where the interior of oil droplets exhibit interfaces, between lipophilic domains and hydrophilic or amphiphilic domains, due to the presence of a lipophilic additive solubilized inside the oil droplets and which is used for delayed release of active elements such that the release of at least one active element, which has a octanol/water partitioning coefficient logP higher than −1, corresponds to a higher Tmax than the Tmax obtained for the simple reference oil-in-water emulsion where no lipophilic additive is used.

According to another feature of the invention, said invention concerns the use of an oil-in-water emulsion where the interior of oil droplets exhibit interfaces, between lipophilic domains and hydrophilic or amphiphilic domains, due to the presence of a lipophilic additive solubilized inside the oil droplets and which is used for delayed release of active elements which have an octanol/water partitioning coefficient logP higher than −1 and which corresponds to a higher Tmax than the Tmax obtained for the simple reference oil-in-water emulsion where no lipophilic additive is used.

The logarithm of the octanol/water partition coefficient (logP) is used extensively to describe the lipophilic or hydrophobic properties of an active element. The logP property value is taken from the ratio of the respective concentrations of the active element in the n-octanol and water partitions of a two-phase system at equilibrium. Among other methods, the classical and most reliable method of logP determination is the shake-flask method, which consists of dissolving some of the active element in question in a volume of n-octanol and water, then measuring the concentration of the solute in each solvent (Organization for Economic Cooperation and Development, Guidelines for The Testing of Chemicals, OECD 107, Partition Coefficient (n-octanol/water)-Shake Flask Method, Adopted 27 Jul. 1995-, OECD, Paris, France).

The Tmax of a given active element released from a given emulsion is a parameter determined from the curve representing the variation over time of the given active element concentration in the water phase or in the headspace of the emulsion or of product containing the emulsion under dynamic condition (denoted as release curve) as time at which the measured concentration of the given active element reaches its maximum (FIG. 1). The analytical methods that can be applied for measurement of release curve and corresponding Tmax in dynamic condition include, but are not restricted to, measuring in headspace under a continuous purging flow of nitrogen and analyzing the active element in the purged headspace gas through its specific mass-to-charge using Proton Transfer Reaction-Mass Spectrometry (PTR-MS) (Pollien, P., Lindinger, C., Yeretzian, C., and Blank, I., “Proton Transfer Reaction Mass Spectrometry, a Tool for On-Line Monitoring of Acrylamide Formation in the Headspace of Maillard Reaction Systems and Processed Food,” Analytical Chemistry, Vol. 75, No. 20, 2003, pp. 5488-5494). The ratio of the Tmax of the oil-in-water emulsion covered by the present invention to the Tmax of the simple reference oil-in-water emulsion is higher than 1.05, more preferably higher than 1.1, more preferably than 1.15, more preferably higher than 1.3 and even more preferably higher than 1.5.

As used herein, a ‘lipophilic additive’ (abbreviated also as ‘LPA’) refers to a lipophilic amphiphilic agent which forms interfaces between lipophilic domains and hydrophilic or amphiphilic domains in a dispersed oil phase. The lipophilic additive (or the mixture of lipophilic additives) is selected from the group consisting of fatty acids, sorbitan esters, propylene glycol mono- or diesters, pegylated fatty acids, monoglycerides, derivatives of monoglycerides, diglycerides, pegylated vegetable oils, polyoxyethylene sorbitan esters, phospholipids, cephalins, lipids, sugar esters, sugar ethers, sucrose esters, polyglycerol esters and mixtures thereof.

The simple reference oil-in-water emulsion is an oil-in-water emulsion with no LPA and where the quantity of LPA present in the oil in water emulsion of the present invention is replaced by the same quantity of oil forming the oil-in-water emulsion of the present invention.

The presence of the LPA inside the oil droplets can result in “self-assembly” structures demonstrating the presence of interfaces, between lipophilic domains and hydrophilic or amphiphilic domains.

Therefore the present invention concerns also the use of an oil-in-water emulsion where the oil droplets exhibit a self-assembled structurization with hydrophilic or amphiphilic domains due to the presence of a lipophilic additive solubilized inside the oil droplets and which is used for delayed release of active elements such that the release of at least one active element, which has a water/octanol partitioning coefficient log p higher than −1, corresponds to a higher Tmax than the Tmax obtained for the simple reference oil-in-water emulsion where no lipophilic additive is used.

The present invention concerns an oil-in-water emulsion where the interior of oil droplets exhibit interfaces, between lipophilic domains and hydrophilic or amphiphilic domains, due to the presence of a lipophilic additive solubilized inside the oil droplets and which is used for delayed release of active elements such that the release of at least one active element, which has a octanol/water partitioning coefficient logP higher than −1, preferably higher than 0, even more prefereably higher then 1, corresponds to a higher Tmax than the Tmax obtained for the simple reference oil-in-water emulsion where no lipophilic additive is used.

The present invention concerns also the use of an oil-in-water emulsion where the oil droplets exhibit a self-assembled structurization with hydrophilic or amphiphilic domains due to the presence of a lipophilic additive solubilized inside the oil droplets and which is used for delayed release of active elements such that the release of at least one active element, which has a water/octanol partitioning coefficient log p higher than −1, preferably higher than 0, even more preferably higher than 1 corresponds to a higher Tmax than the Tmax obtained for the simple reference oil-in-water emulsion where no lipophilic additive is used.

The notion ‘self-assembly’ or ‘self-organization’ refers to the spontaneous formation of aggregates (associates) by separate molecules. Molecules in self-assembled structures find their appropriate location based solely on their structural and chemical properties due to given intermolecular forces, such as hydrophobic, hydration or electrostatic forces (Evans, D. F.; Wennerström, H. (Eds.); ‘The Colloidal Domain’, Wiley-VCH, New York, (1999)). The result of self-assembly does not depend on the process of preparation itself and corresponds to a state of minimum energy (stable equilibrium) of the system.

The invention is directed to the delivery of active elements, which will interact with interfaces, between lipophilic domains and hydrophilic or amphiphilic domains, created by the presence of the LPA, changing their release characteristic depending on the type of molecules and on the octanol/water partitioning coefficient. The invention is directed to the delayed release of at least one active elements. The amount of the active element is higher than 0.0001 PPM of the total composition and lower than 80% of the total composition.

The amount of the active element is preferably higher than 0.0001 PPM of the total composition and lower than 20% of the total composition.

The oil-in-water emulsions of this invention have oil droplets of a diameter in the range of 5 nm to hundreds of micrometers.

The LPA can be added as such or made in-situ by chemical, biochemical, enzymatic or biological means. The amount of oil droplets present in the emulsion of this invention (oil droplet volume fraction) is the amount generally used in ordinary oil-in-water emulsion products. It can vary between 0.00001 wt % and 80 wt %. The oil-in-water emulsion of the invention can be either an oil-in-water emulsion (larger oil droplets), a o/w minie-emulsion, a o/w nano-emulsion or an o/w microemulsion, depending on the size of the oil droplets.

The oil-in-water emulsion of this invention comprises dispersed oil droplets having interfaces, between lipophilic domains and hydrophilic or amphiphilic domains, created by the lipophilic additives and comprising
(i) an oil selected from the group of consisting of mineral oils, hydrocarbons, vegetable oils, waxes, alcohols, fatty acids, mono-, di-, tri-acylglycerols, essential oils, flavouring oils, lipophilic vitamins, esters, neutraceuticals, terpins, terpenes and mixtures thereof.
(ii) a lipophilic additive (LPA) or mixtures of lipophilic and hydrophilic additives, having a resulting HLB value (Hydrophilic-Lipophilic Balance) lower than about 10,
(iii) hydrophilic or amphiphilic domains in form of droplets or channels comprising of water or a non-aqueous polar liquid, such as a polyol.
   and
   an aqueous continuous phase, which contains a hydrophilic emulsifier.

The oil is taken in the large sense. It can be liquid or solid.

The lipophilic additive (LPA) can also be mixed with a hydrophilic additive (having a HLB larger than 10) up to the amount that the mixture is not exceeding the overall HLB of the mixture of 10 or preferably 8. The additive (mixture) can also be made in-situ by chemical, biochemical, enzymatic or biological means.

The amount of added lipophilic additive is defined as α. α is defined as the ratio LPA/(LPA+oil)×100. α is preferably higher than 0.1, more preferably higher than 0.5, even more preferably higher than 1, even more preferably higher than 2, even more preferably higher than 3.

The ratio α=LPA/(LPA+oil)*100 is preferably lower than 99.9, more preferably lower than 99.5, even more preferably lower than 99.0, even more preferably lower than 95, even more preferably lower than 84, even more preferably lower than 80 and most preferably lower than 70. Any combination of the lower and upper range is comprised in the scope of the present invention. α can be given either in wt-% or mol-%. The lower and higher limit of α depends on the properties of the taken oil and LPA, such as the polarity, the molecular weight, dielectric constant, etc., or physical characteristics such as the critical aggregation concentration (cac) or the critical micellar concentration (cmc) of the LPA in the oil droplet phase.

In the present invention, the active element can be taken from the group consisting of flavors, flavor precursors, aromas, aroma precursors, taste enhancers, salts, sugars, amino-acids, polysaccharides, enzymes, peptides, proteins or carbohydrates, food supplements, food additives, hormones, bacteria, plant extracts, medicaments, drugs, nutrients, chemicals for agro-chemical or cosmetical applications, carotenoids, vitamins, antioxidants or nutraceuticals selected from the group comprising of lutein, lutein esters, β-carotene, tocopherol, tocopherol acetate, tocotrienol, lycopene, Co-Q₁₀, flax seed oil, fish oil, omega-3 oils, omega-6 oils, DHA, EPA, arachidonic-rich oils, LCPUFA oils, menthol, mint oil, lipoic acid, vitamins, polyphenols and their glycosides, ester and/or sulfate conjugates, isoflavones, flavonols, flavanones and their glycosides such as hesperidin, flavan 3-ols comprising catechin monomers and their gallate esters such as epigallocatechin gallate and their procyanidin oligomers, vitamin C, vitamin C palmitate, vitamin A, vitamin B₁₂, vitamin D, α- and γ-polyunsaturated fatty acids, phytosterols, esterified phytosterol, non esterified phytosterol, zeaxanthine, caffeine, and a combination thereof.

The active elements can be oil-soluble, oil non-soluble, water soluble or crystallinic.

In the present invention the active element can also be an oil or a LPA.

In the present invention, the LPA is selected from the group of long-chain alcohols, fatty acids, pegylated fatty acids, glycerol fatty acid esters, monoglycerides, diglycerides, derivatives of mono-diglycerides, pegylated vegetable oils, sorbitan esters, polyoxyethylene sorbitan esters, propylene glycol mono- or diesters, phospholipids, phosphatides, cerebrosides, gangliosides, cephalins, lipids, glycolipids, sulfatides, sugar esters, sugar ethers, sucrose esters, sterols, polyglycerol esters. Preferably the LPA is selected from the group consisting of myristic acid, oleic acid, lauric acid, stearic acid, palmitic acid, PEG 1-4 stearate, PEG 2-4 oleate, PEG-4 dilaurate, PEG-4 dioleate, PEG-4 distearate, PEG-6 dioleate, PEG-6 distearate, PEG-8-dioleate, PEG-3-16 castor oil, PEG 5-10 hydrogenated castor oil, PEG 6-20 corn oil, PEG 6-20 almond oil, PEG-6 olive oil, PEG-6 peanut oil, PEG-6 palm kernel oil, PEG-6 hydrogenated palm kernel oil, PEG-4 capric/caprylic triglyceride, mono, di, tri, tetraesters of vegetable oil and sorbitol, pentaerythrityl di, tetra stearate, isostearate, oleate, caprylate or caprate, polyglyceryl-3 dioleate, stearate, or isostearate, plyglyceryl 4-10 pentaoleate, polyglyceryl 2-4 oleate, stearate, or isostearate, polyglyceryl 4-10 pentaoleate, polyglycewryl-3 dioleate, polyglyceryl-6 dioleate, polyglyceryl-10 trioleate, polyglyceryl-3 distearate propylene glycol mono- or diesters of C₆ to C₂₀ fatty acid, monoglycerides of C₆ to C₂₀ fatty acid, lactic acid derivatives of monoglycerides, lactic acid derivatives of diglycerides, diacetyl tartaric ester of monoglycerides, triglycerol monostearate cholesterol, phytosterol, PEG 5-20 soya sterol, PEG-6 sorbitan tetra, hexasterarate, PEG-sorbitan tetraoleate, sorbitan monolaurate, sorbitan monopalmitate, sorbitan mono trioleate, sorbitan mono and tristearate, sorbitan monoisostearate, sorbitan sesquioleate, sorbitan sesquistearate, PEG-2-5 oleyl ether, POE 2-4 lauryl ether, PEG-2 cetyl ether, PEG-2 stearyl ether, sucrose distearate, sucrose dipalmitate, ethyl oleate, isopropyl myristate, isopropyl palmitate, ethyl linoleate, isopropyl linoleate, poloxamers, phospholipids, lecithins, cephalins, oat lipids and lipophilic amphiphilic lipids from other plants; and mixtures thereof.

The oil-in-water emulsion of this invention is stabilized by a hydrophilic emulsifier suitable to stabilize ordinary oil-in-water emulsion droplets. The hydrophilic emulsifier can also be denoted “secondary emulsifier” or “stabilizer”. The emulsion can be aggregated (flocculated) or not depending on the used hydrophilic emulsifier. The hydrophilic emulsifier is selected from the group consisting of low molecular weight hydrophilic surfactants having a HLB>8, gelatin, proteins from e.g. milk (whey protein isolate, caseinate) or soya, block co-polymers, surface active hydrocolloids such as gum arabic, diblock-copolymer or apoprotein-like biopolymers, such as protein-polysaccaride conjugates or coacervates, or protein-polysaccharide, protein-protein, or polysaccharide-polysaccharide hybrids, conjugates or coacervates or mixtures of polymers and biopolymers. Particles (nano or micro) can also be used to stabilize the oil-in-water emulsion of this invention.

The main consideration of emulsion technologists concerns the selection of surface active ingredients, also denoted as surfactants or emulsifiers, which show good surface properties (or activity), i.e., an effective adsorption to the interface formed around the oil droplets, and an effective and efficient reduction of the interfacial tension. The lower the interfacial tension between the aqueous phase and the oil phase gets, the less energy is needed to increase the water-oil interfacial area, i.e., the easier it is to make smaller oil droplets and more stable emulsions.

The hydrophilic emulsifier can also be mixed with the LPA, or with the oil, or with the LPA and the oil. This means, that the hydrophilic emulsifier can partly also be present in the interior of the oil droplet and affecting the internal structure and the interfaces in the oil droplet.

The ratio β=hydrophilic emulsifier/(LPA+oil)×100 describes the amount of hydrophilic emulsifier used to stabilize the oil droplets with respect to the oil plus LPA content. β is preferably higher than 0.1, more preferably higher than 0.5, more preferably higher than 1, and more preferably higher than 2.

The ratio β=hydrophilic emulsifier/(LPA+oil)×100 is preferably lower than 90, more preferably lower than 75 and even more preferably lower than 50. Any combination of the lower and upper range is comprised in the scope of the present invention. β can be given either in wt-% or mol-%. In certain cases the hydrophilic emulsifier is added to the formulation. In other cases, the hydrophilic emulsifier can be present in the product itself such as a food product, a cream, etc and in this case, it is not necessary to add it. An example is milk where the proteins already present can be used as hydrophilic emulsifier of the oil-in-water emulsion of this invention.

In the present invention, the emulsifier can be also selected from the group consisting of low molecular weight surfactants having a HLB>8, proteins from milk, such as whey proteins, whey protein isolates, whey protein concentrates, whey protein aggregates, caseinates, casein micelles, caseins, lysozyme, albumins, or proteins from soya, or amino acids peptides, protein hydrolysates, block co-polymer, random co-polymers, Gemini surfactants, surface active hydrocolloids such as gum arabic, xanthan gum, gelatin, polyelectrolytes, carrageenans, caboxymethylcellulose, cellulose derivatives, Acacia gum, galactomannans, chitosans, hyaluronic acid, pectins, propylene glycol alginate, modified starches, Portulaca Oleracea, Tragacanth, gellan gum, apoprotein-like biopolymers, such as protein-polysaccharide conjugates or coacervates, or protein-polysaccharide, protein-protein, or polysaccharide-polysaccharide hybrids, conjugates, or mixtures of polymers and biopolymers, polyelectrolyte-surfactant complexes, DNA, nucleic acid, particles (micro or nano-sized), starch and starch-based polymers, amylose, amylopectin and mixtures thereof.

The invention concerns the use of the oil-in-water emulsion for delayed release of active elements during storage, consumption or digestion.

The invention concerns the use of the oil-in-water emulsion for delayed release in the mouth.

The oil-in-water emulsion of this invention can be dried and can be in a powder form.

The oil-in-water emulsion according to the invention can be a final product.

The oil-in-water emulsion according to the invention can also be an intermediate product or an additive to a final product.

The oil-in-water emulsion according to the invention is normally in liquid or semi-liquid form. According to another embodiment of the invention, the emulsion is dried and is available in powder form. The oil-in-water emulsion according to the invention is either a final product or an additive. The amount of the additive in the final product is not critical and can be varied.

The emulsion, for controlling the release of molecules, described in this invention is different from ordinary oil-in-water or water-in-oil-in-water double emulsions, including nano- and microemulsions, in which the oil droplets do not have LPA and interfaces, between lipophilic domains and hydrophilic or amphiphilic domains inside the oil droplets. The droplets basically consist of oil droplets which have interfaces with hydrophilic or amphiphilic domains.

It is, therefore, an object of this invention to provide a new oil-in-water emulsion formulation which can be used for delaying the release of active elements in order to deliver new sensation or new nutritional impact or new delivery systems for drugs.

The present invention can be used not only for controlled release of active elements present in food products, but also to products produced in other Industries, such as, Pet Food, Neutraceuticals, Functional Food, Detergents, Nutri-cosmeticals, Cosmetics, Pharmaceuticals, Drug Delivery, Paints, Medical or Agro-chemical Industry, Explosives, Textiles, Mining, Oil well drilling, Paints, Paper Industry, Polymer Industry.

According to the invention, the formation of the interfaces between lipophilic domains and hydrophilic or amphiphilic domains inside the oil droplets of the oil-in-water emulsion of this invention can be realised in different ways. One way is to add a lipophilic additive (LPA) that allows the spontaneous formation of interfaces, to the oil phase prior to the homogenisation step. The other way is to add the lipophilic additive (LPA) to the emulsion product after the homogenisation step. In this case the lipophilic additive will dissolve into the oil droplets and will lead to the formation of the interfaces inside the oil droplets. As homogeniser, an ordinary industrial or lab-scale homogeniser, such as a Rannie piston homogeniser, a Kinematica rotor stator mixer, a colloid mill, a Stephan mixer, a Couette shear cell or a membrane emulsification device can be taken. Moreover, ultrasound, steam injection or a kitchen mixer are also suitable to produce the emulsion described in this invention. The spontaneous formation of the interfaces inside the oil droplets is independent on the energy intake, used to make the emulsion, and the sequence of LPA addition. This means that also Nano and Microfluidics technics are suitable to make the emulsion of this invention.

Heating may also facilitate the dispersion process since the internal structure at high temperatures may be less viscous and the dispersion process may require less shear forces at higher temperatures than at lower temperatures.

Another route for making the emulsion of this invention is the use of hydrotropes or water structure breakers, or spontaneous emulsification which can be chemically or thermodynamically driven (Evans, D. F.; Wennerström, H. (Eds.); ‘The Colloidal Domain’, Wiley-VCH, New York, (1999)).

Another route for making the emulsion of this invention is by combining the spontaneous formation of the interfaces inside the oil droplets of the oil-in-water emulsion with the spontaneous formation of the oil droplets, i.e., the entire emulsion of this invention, by adding diblock-copolymer or apoprotein-like biopolymers, such as protein-polysaccharide conjugates or coacervates or protein-polysaccharide, protein-protein, or polysaccharide-polysaccharide hybrids or mixtures of polymers or biopolymers or hydrophilic low molecular weight surfactants.

Another route for making the emulsion of this invention is to use dialysis. One way is to mix the lipophilic additive (LPA) to the oil phase and to the hydrophilic emulsifier, used to stabilize the oil droplets in the emulsion. The mixture consisting of the LPA, the oil phase and the hydrophilic emulsifier are mixed with water in such a way that a micellar or lamellar or any other phase is formed. Using a dialysis membrane enables to remove the excess of the hydrophilic emulsifier in the bulk aqueous phase and the oil-in-water emulsion of this invention is formed.

Another route for making the emulsion of this invention is to use the control action of a guest molecule to modify the internal structure of the oil droplets of this invention in such a way that the oil droplet phase is less viscous and requires less energy to be dispersed into the aqueous phase than the droplet phase consisting of the oil-LPA-water and no guest molecule. Dispersing the concentrated mixture (oil-LPA-Guest molecule-water) will be easy since the oil phase structure is of low viscosity. The internal structure of the oil droplets of the emulsion changes upon dilution since guest molecules leave the oil droplets and dissolves into the aqueous continuous phase during homogenisation and dilution. For this route, the guest molecule is preferably hydrophilic and osmotically active.

### BRIEF DESCRIPTION OF THE DRAWINGS

Table 1 presents the information about aroma compounds used as active elements in the examples 3 to 6.

FIG. 1 shows two theoretical release curves of a given active element from two oil-in-water emulsions, A and B, with corresponding parameters: the maximum concentration Cmax(A), the time to reach maximum concentration Tmax (A) and Cmax(B), Tmax (B). In this figure, the oil-in-water emulsion B shows a delayed release of the active element compared to the oil-in-water emulsion A: illustrated by a Cmax(B) lower than Cmax(A) and notably a Tmax (B) larger than Tmax (A)

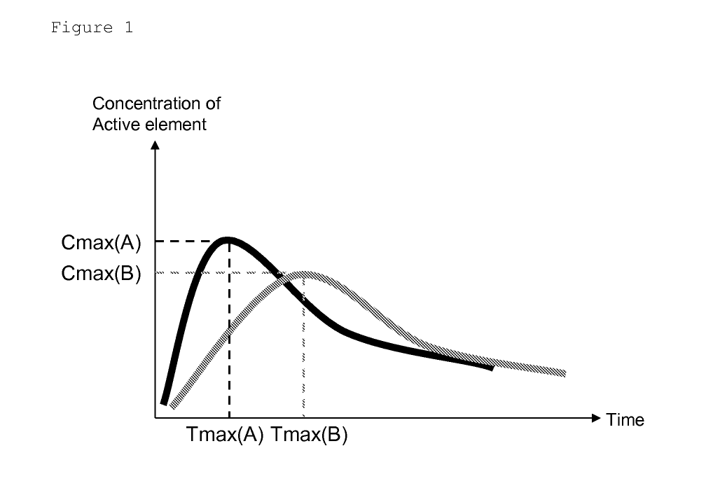

FIG. 2 shows the maximum concentration (Cmax) of nine active elements released under in vitro dynamic condition in the headspace of three emulsions (one emulsion of the present invention and two comparative emulsions).

FIG. 3 shows the time to reach maximum concentration (Tmax) of nine active elements released under in vitro dynamic condition in the headspace of three emulsions (one emulsion of the present invention and two comparative emulsions).

TABLE 1

| | | | | | Concen- |
|---|---|---|---|---|---|
| | | | | | tration |
| Active | | | | | used |
| elements name | CAS # | Supplier | Product # | logP | (ppm) |
| Diacetyl | 431-03-8 | Aldrich | W237035 | −1.332 | 20 |
| Acetaldehyde | 75-07-0 | Aldrich | W200301 | −0.156 | 10 |
| 2E-hexenal | 6728-26-3 | Aldrich | W256110 | 1.576 | 20 |
| cis-3-Hexen-1-ol | 928-96-1 | Aldrich | W256323 | 1.612 | 20 |
| Benzaldehyde | 100-52-7 | Aldrich | W212717 | 1.64 | 20 |
| Ethyl | 108-64-5 | Aldrich | W246328 | 2.118 | 20 |
| isovalerate | | | | | |
| 3-methoxy-2 | 24683-00-9 | Aldrich | W313203 | 2.622 | 100 |
| isobutylpyrazine | | | | | |
| Octanal | 124-13-0 | Aldrich | W279714 | 3.032 | 100 |
| Linalool | 78-70-6 | Aldrich | W263508 | 3.281 | 100 |

Table 1 lists active elements used in the study, providing chemical name (first column), the Chemical Abstract Service number (CAS#, second column), supplier and supplier product code (third and fourth column), the logarithm of the octanol/water partition coefficient (logP) and the concentration used in final formulations in parts per million of volume (ppmV). These nine active elements are volatile aroma compounds.

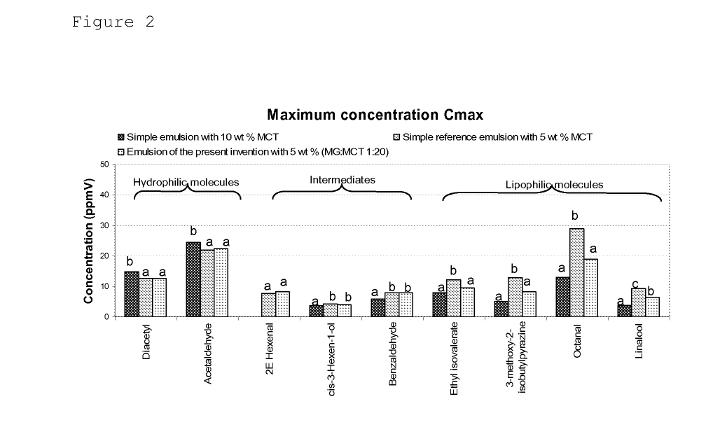

FIG. 2 shows the maximum concentration (Cmax) of nine active elements released in the headspace of three emulsions under in vitro dynamic conditions, monitored by Proton Transfert-Mass Spectrometry (PTR-MS) (Pollien, P., Lindinger, C., Yeretzian, C., and Blank, I., “Proton Transfer Reaction Mass Spectrometry, a Tool for On-Line Monitoring of Acrylamide Formation in the Headspace of Maillard Reaction Systems and Processed Food,” Analytical Chemistry, Vol. 75, No. 20, 2003, pp. 5488-5494). A double-jacketted glass cell was held at 36° C. with a circulating water bath. This cell was put in the oven which was held at 60° C. in order to avoid cold points and water condensation. The headspace cell was continuously purged at 200 sccm (standard cubic centimeters per minute) with pure nitrogen. Prior to introduction into the PTR-MS, the headspace was diluted with 1960 sccm of nitrogen to avoid water saturation of the instrument.

When all the set-up had been thermo-stabilized, 100 ml of sample, after being stabilized at 36° C. in a water bath, was poured inside the cell, which was quickly reconnected and put under agitation at 135 rpm. The release of active elements was monitored on-line during 10 minutes for each sample.

Nine specific masses were selected based on the scan data, i.e., m/z 45 for acetaldehyde, m/z 83 for cis-3-Hexen-1-ol, m/z 87 for diacetyl, m/z 99 for 2E-hexenal, m/z 107 for benzaldehyde, m/z 111 for octanal, m/z 131 for ethyl isovalerate, m/z 137 linalool, m/z 167 for 3-methoxy-2-isobutylpyrazine.

The PTR-MS signals (ion count) enabled us to calculate the active element concentration in the headspace of the emulsion so that to build the release curve for the active element. Cmax of a given active element released from a given emulsion was determined from the release curve as the maximum concentration of the active element released in the headspace of the emulsion, expressed in ppmV (parts per million in volume).

In the figure, the nine active elements are listed in X axis and sorted along their logP, from the lowest logP value (hydrophilic compounds) to the highest logP (lipophilic compounds). Black, grey and hatched bars represent respectively Cmax released in the headspace of an oil-in-water emulsion containing 10 wt % Medium Chain Triglycerides (MCT) (herein labelled as simple emulsion with 10 wt % MCT), of an oil-in-water emulsion containing 5 wt % MCT (herein labelled as simple reference emulsion with 5 wt % MCT), and of an oil-in-water emulsion containing 5 wt % of an 1:20 mixture of a unsaturated monoglyceride (DIMODAN MO90, Danisco, Denmark) and MCT (herein Emulsion of the present invention with 5 wt % (MG:MCT 1:20)). All Cmaxvalues were obtained on 6 replicated measurements. Cmax of 2E-Hexenal for simple emulsion with 10 wt % MCT is not determined because its concentration in the headspace was continuously and gradually increasing over the 10 minutes of measurement. The letter (a), (b) and (c) over each bar represent result of statistical analysis of variance and post-hoc comparison test using test Fisher Least Significant Difference (James E. D Muth, 1999, Basic Statistics and Pharmaceutical Statistical Applications, Marcel Dekker publication, 596 pp): for each active element, two bars identified with different letters are representing Cmax values which are significantly different (with a first order statistical risk, α, inferior to 0.05). The results show that the simple reference emulsion with 5 wt % MCT exhibits a significantly higher maximum concentration (Cmax) released in the headspace for intermediate and lipophilic active elements and lower Cmax for hydrophilic compounds compared to the simple emulsion with 10 wt % MCT. FIG. 2 also indicates that Cmax of lipophilic active elements released in the headspace of the emulsion of the present invention with 5 wt % (MG:MCT 1:20) is significantly lower than that of the simple reference emulsion with 5 wt % MCT and has no difference with that of the simple emulsion with 10 wt % MCT, excepted that Cmax of linalool is still significantly higher for the emulsion of the present invention with 5 wt % (MG:MCT 1:20) than for the simple emulsion 10 wt % MCT. No difference in Cmax of hydrophilic and intermediate active elements is demonstrated between the emulsion of the present invention with 5 wt % (MG:MCT 1:20) and the simple reference emulsion with 5 wt % MCT. We can conclude that lipophilic active elements are released with a larger maximum concentration when MCT oil is reduced from 10 wt % to 5 wt %. The emulsion of the present invention with 5 wt % (MG:MCT 1:20), with the same overall oil level than the simple reference emulsion with 5 wt % MCT but with the presence of the lipophilic additive, lowers the release of lipophilic active elements and therefore moves the kinetic of active element release closer to that of the simple emulsion with 10 wt % MCT.

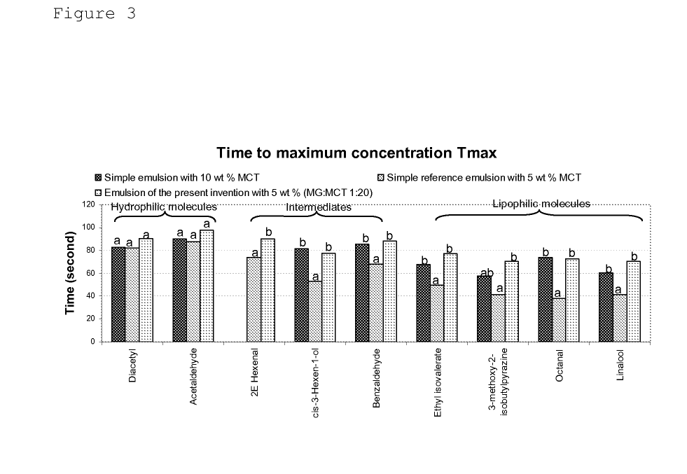

FIG. 3 shows the time to reach maximum concentration (Tmax) of nine active elements released in the headspace of three emulsions under in vitro dynamic condition, monitored by PRT-MS as explained in the FIG. 2. Tmax of a given active element released from a given emulsion was determined from the release curve as the time to reach maximum concentration of the active element released in the headspace of the emulsion, expressed in seconds. The nine active elements are listed in X axis and sorted along their logP, from the lowest logP value (hydrophilic compounds) to the highest logP (lipophilic compounds). Black, grey and hatched bars represent respectively Tmax the active elements released in the headspace of an oil-in-water emulsion containing with 10 wt % Medium Chain Triglycerides (MCT) (herein labelled as simple emulsion with 10 wt % MCT), of an oil-in-water emulsion containing 5 wt % MCT (herein labelled as simple reference emulsion with 5 wt % MCT), and an oil-in-water emulsion containing 5 wt % of an 1:20 mixture of a unsaturated monoglyceride (DIMODAN MO90, Danisco, Denmark) and MCT (herein Emulsion of the present invention with 5 wt % (MG:MCT 1:20)). All Tmax values were obtained on 6 replicated measurements. Tmax of 2E-Hexenal for the simple emulsion with 10 wt % MCT is not determined because its concentration in the headspace was continuously and gradually increasing over the 10 minutes of measurement. The letter (a), (b) and (c) over each bar represent result of statistical analysis of variance and post-hoc comparison test using test Fisher Least Significant Difference (James E. D Muth, 1999, Basic Statistics and Pharmaceutical Statistical Applications, Marcel Dekker publication, 596 pp): for each active element, two bars identified with different letters are representing Tmax values which are significantly different (with a first order statistical risk, α, inferior to 0.05). A significant lower time to reach the maximum concentration (Tmax) is observed for intermediate and lipophilic compounds released from the simple reference emulsion with 5 wt % MCT than from the simple emulsion with 10 wt % MCT, however, there is no significant difference in Tmax for hydrophilic compounds between these two emulsions. Comparing the emulsion of this invention with the 5 wt % (MG:MCT 1:20) to the simple reference emulsion 5 wt % MCT, Tmax is increased by a factor of 1.1 and 1.12 respectively for diacetyl and acetaldehyde (hydrophilic active elements), which leads to a non-significant difference, and increased by a factor of 1.46, 1.30, 1.55, 1.7, 1.91 and 1.7 respectively for cis-3-hexen-1-ol, benzaldehyde, ethyl isovalerate, 3-methoxy-2-isobutylpyrazine, octanal et linalool (intermediate and lipophilic active elements, which leads to a significant increase. FIG. 3 also reveals no significant difference in Tmax between the emulsion of this invention with 5 wt % (MG:MCT 1:20) and the simple emulsion with 10 wt % MCT for all active elements. We can conclude that lipophilic active elements are released earlier when the oil content of a simple emulsion is reduced from 10 wt % to 5 wt %, and that the emulsion of this invention with 5 wt % (MG:MCT 1:20), with the same overall oil level as the simple emulsion with 5 wt % MCT and the presence of the lipophilic additive, delays the release of lipophilic active elements and therefore moves the kinetic of active element release closer to that of the simple emulsion with 10 wt % MCT.

### EXAMPLES

### Example 1

This example covers the invention. Preparation of an oil in water emulsion where the oil droplets are structured according to the invention.

### Materials Used to Make the Oil in Water Emulsion

The Epikuron 200 used, was purchased from Degussa (Hamburg, Germany) and is a purified soya phosphatidylcoline (SPC) with a linoleic acid content of more than 60% of the total fatty acid content, the rest of the acyl chains are mainly palmitoyl and oleoyl chains. SPC is semicrystalline and contains 1-2 moles of crystal water.

The Diacylglycerol (DAG) rich in diolein used, was Glycerol Dioleate, and was supplied by Danisco(Brabrand, Denmark). It contains 95.3% of diglycerides, 4.0% triglycerides, 0.5% monoglycerides and 0.1% of free fatty acids. We used Ethanol absolute GR for analysis, 99.9% pure and obtained from Merck KGaA (Darmstadt, Germany).

Water used was Milli-Q water.

The Soyabean oil fully refined, with more than 0.1% of fully fatty acid was provided from Nutriswiss (Lyss, Switzerland).

As stabilizers, we use Tween 80 from Fluka Chemie Gmbh (Burchs, Switzerland).

### Preparation of the Stock Solution Used to Make the Oil in Water Emulsion

The different non dispersed samples were prepared individually by weighing the appropriate amounts of the substances Epikuron 200 and Diacylglycerol into 18×100 mm Pyrex tubes and then adding approximately 0.5 ml (plastic pipette) of the organic solvent ethanol, in order to help solubilise the Epikuron 200. They were then heated (approximately 90° C.) in a block heater (URB, Grant, UK) and homogenized by vigorous agitation with a Vortex (Bender & Hobein AG, Zurich, Switzerland) until total solubilisation of the Epikuron 200. The ethanol was removed from the samples by using nitrogen.

The samples were then left to cool at room temperature.

After this step, the samples were again heated, approximately to 90° C., the water was added and the temperature was increased to 100° C. during 5 minutes, followed by vigorous stirring with a Vortex.

The samples were then let cooled to room temperature.

For a better conduction of the heat between the glass tube and the bulk solution, during all this procedure we wrapped up the tube in aluminium paper.

### Dispersion Procedure to Obtain the Oil in Water Emulsion

In our study, the aqueous dispersions were prepared by weighing the appropriate amounts of water and stabilizer in a 25 ml beaker, mixing it by magnetic agitation, until total solubilisation of the stabilizer. This solution was added to a mixture of a certain amount of stock solution.

In order to form the dispersion, this mixture was treated by ultrasonication in a High-Intensity Ultrasonic Processor (UP400S. Hielscher, Ultrasound Technology, Germany) at 70% of the maximum amplitude power in cycles of 1 for approximately 1 or 2 minutes, resulting in a milky dispersion. Some other samples used ultrasound conditions of 30% amplitude in cycles of 0.5 during 20 minutes.

Obtention of an Oil in Water Emulsion where the Oil Droplets are Structured According to the Invention and Contains Phospholipids, Diacylglycerol (DAG), Tween 80 and Water.

Using the procedure, described previously to obtain oil in water emulsions, a dispersion was obtained where the final composition is: 0.407% Epikuron 200, 0.613% DAG, 0.0999% Tween80 and 98.881% water. The oil droplets containing Phospholipids (Epikuron 200), diacylglycerol and Tween 80 are structured by the phospholipids and exhibit interfaces, between lipophilic domains, hydrophilic or amphiphilic domains.

### Example 2

This example covers the invention. Obtention of an oil in water emulsion where the oil droplets are structured according to the invention and contains Phospholipids, triglycerides, Diacylglycerol (DAG), Tween 80 and water.

Using the procedure, described in example 1, a dispersion is obtained where the final composition is 0.294% Phospholipid (Epikuron 200), 0.595% DAG, 0.115% Soyabean oil, 98.886% water and 0.110% Tween 80. The oil droplets containing Phospholipids (Epikuron 200), diacylglycerol, triacylglycerol (Soyabean oil) and Tween 80 are structured by the phospholipids and exhibit interfaces, between lipophilic domains, hydrophilic or amphiphilic domains.

### Example 3

This example covers the invention. Obtention of an oil in water emulsion where the oil droplets are structured according to the invention and containing monoglyceride, medium chain trigleceride and sodium caseinate

An oil-in-water emulsion where the oil droplets exhibit interfaces, between lipophilic domains and hydrophilic or amphiphilic domains, due to the presence of a lipophilic additive solubilized inside the oil droplets containing 5% wt oil was obtained by dispersing the oil mixture of an unsaturated monoglyceride (DIMODAN M090, Danisco, Denmark) with MCT oil in ratio 1:20 wt (MG:MCT 1:20) in sodium caseinate solution 0.8 wt % at temperatures between 50° C. and 60° C. (herein Emulsion of this invention with 5 wt % (MG:MCT 1:20)). The emulsion of the present invention was homogenized under high pressure by Rannie homogenizer. The oil droplets containing monoglyceride, triglyceride and sodium caseinate, exhibit interfaces, between lipophilic domains, hydrophilic or amphiphilic domains.

### Example 4

This example covers the invention.

Nine active elements varying in octanol/water partition coefficient (logP) including diacetyl (logP −1.332), acetaldehyde (−0.156), 2E-hexenal (1.576), cis-3-Hexen-1-ol (1.612), benzaldehyde (1.64), ethyl isovalerate (2.118), 3-methoxy-2-isobutylpyrazine (2.622), octanal (3.032), linalool (3.281) (Table 1) were incorporated into the emulsion of this invention with 5 wt % (MG:MCT 1:20) and which is described in example 3. In the present example, active elements are volatile aroma compounds. The active element-incorporated emulsion of the present invention with 5 wt % (MG:MCT 1:20) sample was stocked in the fridge at 5° C. during 24 hours before the analysis of the release.

This procedure was done in six replicates, leading to 6 samples of active element-incorporated emulsion of the present invention with 5 wt % (MG:MCT 1:20) to be used for active element release measurement.

The active element release from each sample of emulsion of the present invention with 5 wt % (MG:MCT 1:20) was monitored by Proton Transfer Reaction Mass Spectrometry under in vitro dynamic conditions.

The maximum concentration Cmax of a given active element released in the headspace of the emulsion of the present invention with 5 wt % (MG:MCT 1:20) is represented by a hatched bar in the FIG. 2. The time to reach maximum concentration Tmax of a given active element released in the headspace of the emulsion of the present invention with 5 wt % (MG:MCT 1:20) is represented by a hatched bar in the FIG. 3. In these two figures, active elements for which Cmax and Tmax were determined are listed in X axis and sorted along their logP, from the lowest logP (hydrophilic compounds) to the highest logP (lipophilic compounds). The active elements are either solubilized in the oil droplets, at the oil droplet/continuous aqueous phase interface or in the continuous phase. Lipophilic active elements are probably mainly solubilized in the oil droplet except during release where they are transported through the aqueous phase to the air.

### Example 5

### This Example is a Comparative Example

An oil-in-water emulsion containing 5% wt Medium Chain Triglycerides (MCT) (herein labelled as simple reference emulsion with 5 wt % MCT) was made by dispersing MCT oil in sodium caseinate solutions 0.8% wt using Polytron and homogenized under high pressure by Rannie homogenizer. The same nine active elements as listed in example 1, varying in octanol/water partition coefficient logP (Table 1) were incorporated in the simple reference emulsion with 5 wt % MCT. The active element-incorporated simple reference emulsion with 5 wt % MCT sample was stocked in the fridge at 5° C. during 24 hours before the active element release analysis. This procedure was done in six replicates, leading to 6 samples of active element-incorporated simple reference emulsion with 5% wt MCT to be used for release measurement.

The active element release from the simple reference emulsion with 5 wt % MCT samples was monitored by Proton Transfer Reaction Mass Spectrometry PTR-MS under in vitro dynamic conditions.

The maximum concentration Cmaxof a given active element released in the headspace of the simple reference emulsion with 5 wt % MCT was represented by a grey bar in the FIG. 2. The time to reach maximum concentration (Tmax) of a given active element released in the headspace of the simple reference emulsion with 5 wt % MCT was represented by a grey bar in the FIG. 3.

### Example 6

### This Example is a Comparative Example

An oil-in-water emulsion containing 10% wt Medium Chain Triglycerides (MCT) (herein labelled as simple emulsion with 10 wt % MCT) was made by dispersing MCT oil in sodium caseinate solutions 1.6% wt using Polytron and homogenized under high pressure by Rannie homogenizer.

The same nine active elements as listed in example 1, varying in octanol/water partition coefficient logP (Table 1) were incorporated in the simple emulsion with 10 wt % MCT. The active element-incorporated simple emulsion with 10 wt % MCT sample was stocked in the fridge at 5° C. during 24 hours before the active element release analysis. This procedure was done in six replicates, leading to 6 samples of active element-incorporated simple emulsion with 10% wt MCT to be used for active element release measurement.

The release from the simple emulsion with 10 wt % MCT samples was monitored by Proton Transfer Reaction Mass Spectrometry PTR-MS under in vitro dynamic conditions.

The maximum concentration Cmax of a given active element released in the headspace of the simple emulsion with 10 wt % MCT was represented by a black bar in the FIG. 2. The time to reach maximum concentration (Tmax) of a given active element released in the headspace of the simple emulsion with 10 wt % MCT was represented by a black bar in the FIG. 3.

1. A method of providing for the delay release of an active element comprising using an oil-in-water emulsion where an interior of oil droplets exhibit interfaces, between lipophilic domains and hydrophilic or amphiphilic domains, due to the presence of a lipophilic additive solubilized inside the oil droplets and which is used for a delayed release of active elements such that the release of at least one active element, which has an octanol/water partitioning coefficient logP greater than −1, corresponds to a greater Tmax than a Tmax obtained for a standard oil-in-water emulsion where no lipophilic additive is used.

2. A method of providing for the delay release of an active element comprising using an oil-in-water emulsion where an interior of oil droplets exhibit interfaces, between lipophilic domains and hydrophilic or amphiphilic domains, due to the presence of a lipophilic additive solubilized inside the oil droplets, and which is used for a delayed release of active elements which have an octanol/water partitioning coefficient logP greater than −1 and which corresponds to a greater Tmax than a Tmax obtained for a standard oil-in-water emulsion where no lipophilic additive is used.

3. A method of providing for the delay release of an active element comprising using an oil-in-water emulsion where the oil droplets exhibit a self-assembled structurization with hydrophilic or amphiphilic domains due to the presence of a lipophilic additive solubilized inside the oil droplets and which is used for a delayed release of active elements such that the release of at least one active element, which has a water/octanol partitioning coefficient logP greater than −1, corresponds to a greater Tmax than a Tmax obtained for a standard oil-in-water emulsion where no lipophilic additive is used.

4. Method according to claim 1 which is used for the delayed release of active elements such that the release of at least one active element, which has a octanol/water partitioning coefficient logP greater than 0, corresponds to a greater Tmax than the Tmax obtained for a standard oil-in-water emulsion where no lipophilic additive is used.

5. Method according to claim 1 wherein the Tmax is increased by a factor greater than 1.15 compared to the Tmax measured from a standard oil-in-water emulsion containing the same oil content but where no lipophilic additive is used.

6. Method according to claim 1 wherein the oil droplets have a diameter of 5 nm to hundreds of micrometers and the oil-in-water emulsion contains the active element which is present at between 0.0001 part per million (ppm) and 80% based on the total composition.

7. Method according to claim 1 comprising dispersed oil droplets having interfaces, between lipophilic domains and hydrophilic or amphiphilic domains, created by the lipophilic additives and comprising:
an oil selected from the group consisting of mineral oils, hydrocarbons, vegetable oils, waxes, alcohols, fatty acids, mono-, di-, tri-acylglycerols, essential oils, flavouring oils, lipophilic vitamins, esters, neutraceuticals, terpins, terpenes and mixtures thereof;
a lipophilic additive (LPA) or mixtures of lipophilic and hydrophilic additives, having a resulting HLB value (Hydrophilic-Lipophilic Balance) lower than about 10;
hydrophilic or amphiphilic domains in the form of droplets or channels comprising water or a non-aqueous polar liquid, such as a polyol;
and
an aqueous continuous phase, which contains a hydrophilic emulsifier.

8. Method according to claim 1, wherein the active element is selected from the group consisting of flavours, flavour precursors, aromas, aroma precursors, taste enhancers, salts, sugars, amino-acids, polysaccharides, enzymes, peptides, proteins or carbohydrates, food supplements, food additives, hormones, bacteria, plant extracts, medicaments, drugs, nutrients, chemicals for agro-chemical or cosmetical applications, carotenoids, vitamins, antioxidants or nutraceuticals selected from the group comprising of lutein, lutein esters, β-carotene, tocopherol, tocopherol acetate, tocotrienol, lycopene, Co-Q₁₀, flax seed oil, fish oil, omega-e oils, omega-6 oils, DHA, EPA, arachidonic-rich oils, LCPUFA oils, menthol, mint oil, lipoic acid, vitamins, polyphenols and their glycosides, ester and/or sulphate conjugates, isoflavones, flavonols, flavanones and their glycosides, flavan 3-ols comprising catechin monomers and their gallate esters, vitamin C, vitamin C palmitate, vitamin A, vitamin B₁₂, vitamin D, α- and γ-polyunsaturated fatty acids, phytosterols, esterified phytosterol, non esterified phytosterol, zeaxanthine, caffeine, and a combination thereof.

9. Method according to claim 1, wherein the LPA is selected from the group consisting of long-chain alcohols, fatty acids, pegylated fatty acids, glycerol fatty acid esters, monoglycerides, diglycerides, derivatives of mono-diglycerides, pegylated vegetable oils, sorbitan esters, poloxyethylene sorbitan esters, propylene glycol mono- or diesters, phospholipids, phosphatides, cerebrosides, gangliosides, cephalins, lipids, glycolipids, sulfatides, sugar esters, sugar ethers, sucrose esters, sterols, and polyglycerol esters.

10. Method according to claim 1, wherein the LPA is selected from the group consisting of myristic acid, oleic acid, lauric acid, stearic acid, palmitic acid, PEG 1-4 stearate, PEG 2-4 oleate, PEG-4 dilaurate, PEG-4 dioleate, PEG-4 distearate, PEG-6 dioleate, PEG-6 distearate, PEG-8 dioleate, PEG-3-16 castor oil, PEG 5-10 hydrogenated castor oil, PEG 6-20 corn oil, PEG 6-20 almond oil, PEG 6 olive oil, PEG-6 peanut oil, PEG-6 palm kernel oil, PEG-6 hydrogenated palm kernel oil, PEG-4 capric/caprylic triglyceride, mono, di, tri, tetraesters of vegetable oil and sorbitol, pentaerythrityl di, tetra stearate, isostearate, oleate, caprylate or caprate, polyglyceryl-e dioleate, stearate, or isostearate, plyglyceryl 4-10 pentaoleate, polyglyceryl 2-4 oleate, stearate, or isostearate, polyglyceryl 4-10 pentaoleate, polyglyceryl-3 dioleate, polyglyceryl-6 dioleate, polyclyceryl-10 trioleate, polyglyceryl-3 distearate propylene glycol mono- or diesters of C₆ to C₂₀ fatty acid, monoglycerides of C₆ to C₂₀ fatty acid, lactic acid derivatives of monoglycerides, lactic acid derivatives of diglycerides, diacetyl tartaric ester of monoglycerides, triglycerol monostrearate cholesterol, phytosterol, PEG 5-10 soya sterol, PEG-6 sorbitan tetra, hexasteararate, PEG-6 sorbitan tetraoleate, sorbitan monolaurate, sorbitan monopalmitate, sorbitan mono trioleate, sorbitan mono and tristearate, sorbitan monoisostearate, sorbitan sesquioleate, sorbitan sesquistearate, PEG-2-5 oleyl ether, POE 2-4 lauryl ether, PEG-2 cetyl ether, PEG-2 stearyl ether, sucrose distearate, sucrose dipalmitate, ethyl oleate, isopropyl myristate, isopropyl palmitate, ethyl linoleate, isopropyl linoleate, poloxamers, phospholipids, lecithins, cephalins, oat lipids and lipophilic amphiphilic lipids from other plants; and mixtures thereof.

11. Method according to claim 1, wherein the emulsifier is selected from the group consisting of low molecular weight surfactants having a HLB>8, proteins from milk, proteins from soya, amino acids peptides, protein hydrolysates, block co-polymer, random co-polymers, Gemini surfactants, surface active hydrocolloids polyelectrolyte-surfactant complexes, DNA, nucleic acid, particles (micro or nano-sized), starch and starch-based polymers, amylase, amylopectin and mixtures thereof.

12. Method according to claim 1 for the delayed release of active elements during a state selected from the group consisting of storage, consumption and digestion.

13. Method according to claim 1 for the delayed release in the mouth.

14. Powder comprising an active element comprising an oil-in-water emulsion having an interior of oil droplets that exhibit interfaces, between lipophilic domains and hydrophilic or amphiphilic domains, due to the presence of a lipophilic additive solubilized inside the oil droplets, and providing for the release of at least one active element, which has a octanol/water partitioning coefficient logP greater than −1, that corresponds to a greater Tmax than a Tmax obtained for a standard oil-in-water emulsion where no lipophilic additive is used the oil-in-water emulsion being dried and being in a powder form.

15. Oil-in-water emulsion providing for the delay release of an active element comprising an oil-in-water emulsion having an interior of oil droplets that exhibit interfaces, between lipophilic domains and hydrophilic or amphiphilic domains, due to the presence of a lipophilic additive solubilized inside the oil droplets, and which is used for delayed release of active elements such that the release of at least one active element, which has a octanol/water partitioning coefficient logP greater than −1, corresponds to a greater Tmax than a Tmax obtained for a standard oil-in-water emulsion where no lipophilic additive is used.

16. Oil-in-water emulsion providing for the delay release of an active element comprising an oil-in-water emulsion having an interior of oil droplets that exhibit interfaces, between lipophilic domains and hydrophilic or amphiphilic domains, due to the presence of a lipophilic additive solubilized inside the oil droplets, and which is used for delayed release of active elements such that the release of at least one active element, which has a octanol/water partitioning coefficient logP greater than −1, corresponds to a greater Tmax than a Tmax obtained for a standard oil-in-water emulsion where no lipophilic additive is used that is used as a starting material, an intermediate product or an additive to a final product.

17. Power comprising an active element comprising an oil-in-water emulsion having an interior of oil droplets that exhibit interfaces, between lipophilic domains and hydrophilic or amphiphilic domains, due to the presence of a lipophilic additive solubilized inside the oil droplets, and which is used for delayed release of active elements which have an octanol/water partitioning coefficient logP greater than −1 and which corresponds to a greater Tmax than a Tmax obtained for a standard oil-in-water emulsion where no lipophilic additive is used, the oil-in-water emulsion is dried and is in a powder form.

18. Powder comprising an active element comprising an oil-in-water emulsion having the oil droplets that exhibit a self-assembled structurization with hydrophilic or amphiphilic domains due to the presence of a lipophilic additive solubilized inside the oil droplets and which is used for delayed release of active elements such that the release of at least one active element, which has a water/octanol partitioning coefficient logP greater than −1, corresponds to a greater Tmax than a Tmax obtained for a standard oil-in-water emulsion where no lipophilic additive is used, the oil-in-water emulsion is dried and is in a powder form.

19. Oil-in-water emulsion providing the delay release of an active element comprising an oil-in-water emulsion having an interior of oil droplets that exhibit interfaces, between lipophilic domains and hydrophilic or amphiphilic domains, due to the presence of a lipophilic additive solubilized inside the oil droplets, and which is used for delayed release of active elements which have an octanol/water partitioning coefficient logP greater than −1 and which corresponds to a greater Tmax than a Tmax obtained for a standard oil-in-water emulsion where no lipophilic additive is used.

20. Oil-in-water emulsion providing for the delay release of an active element comprising an oil-in-water emulsion where the oil droplets exhibit a self-assembled structurization with hydrophilic or amphiphilic domains due to the presence of a lipophilic additive solubilized inside the oil droplets and which is used for delayed release of active elements such that the release of at least one active element, which has a water/octanol partitioning coefficient logP greater than −1, corresponds to a greater Tmax than a Tmax obtained for a standard oil-in-water emulsion where no lipophilic additive is used.

21. Oil-in-water emulsion providing the delay release of an active element comprising an oil-in-water emulsion where an interior of oil droplets exhibit interfaces, between lipophilic domains and hydrophilic or amphiphilic domains, due to the presence of a lipophilic additive solubilized inside the oil droplets, and which is used for delayed release of active elements which have an octanol/water partitioning coefficient logP greater than −1 and which corresponds to a greater Tmax than a Tmax obtained for a standard oil-in-water emulsion where no lipophilic additive is used, the oil-in-water emulsion is used as a starting material, an intermediate product or an additive to a final product.

22. Oil-in-water emulsion providing for the delay release of an active element comprising an oil-in-water emulsion where the oil droplets exhibit a self-assembled structurization with hydrophilic or amphiphilic domains due to the presence of a lipophilic additive solubilized inside the oil droplets and which is used for delayed release of active elements such that the release of at least one active element, which has a water/octanol partitioning coefficient logP greater than −1, corresponds to a greater Tmax than a Tmax obtained for a standard oil-in-water emulsion where no lipophilic additive is used, the oil-in-water emulsion is used as a starting material, an intermediate product or an additive to a final product.

23. Method according to claim 2 which is used for the delayed release of active elements such that the release of at least one active element, which has a octanol/water partitioning coefficient logP greater than 0, corresponds to a greater Tmax than the Tmax obtained for a standard oil-in-water emulsion where no lipophilic additive is used.

24. Method according to claim 2 wherein the Tmax is increased by a factor greater than 1.15 compared to the Tmax measured from a standard oil-in-water emulsion containing the same oil content but where no lipophilic additive is used.

25. Method according to claim 2 wherein the oil droplets have a diameter of 5 nm to hundreds of micrometers and the oil-in-water emulsion contains the active element which is present at between 0.0001 part per million (ppm) and 80% based on the total composition.

26. Method according to claim 2 comprising dispersed oil droplets having interfaces, between lipophilic domains and hydrophilic or amphiphilic domains, created by the lipophilic additives and comprising:
an oil selected from the group consisting of mineral oils, hydrocarbons, vegetable oils, waxes, alcohols, fatty acids, mono-, di-, tri-acylglycerols, essential oils, flavouring oils, lipophilic vitamins, esters, neutraceuticals, terpins, terpenes and mixtures thereof;
a lipophilic additive (LPA) or mixtures of lipophilic and hydrophilic additives, having a resulting HLB value (Hydrophilic-Lipophilic Balance) lower than about 10;
hydrophilic or amphiphilic domains in the form of droplets or channels comprising water or a non-aqueous polar liquid, such as a polyol;
and
an aqueous continuous phase, which contains a hydrophilic emulsifier.

27. Method according to claim 2, wherein the active element is selected from the group consisting of flavours, flavour precursors, aromas, aroma precursors, taste enhancers, salts, sugars, amino-acids, polysaccharides, enzymes, peptides, proteins or carbohydrates, food supplements, food additives, hormones, bacteria, plant extracts, medicaments, drugs, nutrients, chemicals for agro-chemical or cosmetical applications, carotenoids, vitamins, antioxidants or nutraceuticals selected from the group comprising of lutein, lutein esters, β-carotene, tocopherol, tocopherol acetate, tocotrienol, lycopene, Co-Q₁₀, flax seed oil, fish oil, omega-e oils, omega-6 oils, DHA, EPA, arachidonic-rich oils, LCPUFA oils, menthol, mint oil, lipoic acid, vitamins, polyphenols and their glycosides, ester and/or sulphate conjugates, isoflavones, flavonols, flavanones and their glycosides, flavan 3-ols comprising catechin monomers and their gallate esters, vitamin C, vitamin C palmitate, vitamin A, vitamin B₁₂, vitamin D, α- and γ-polyunsaturated fatty acids, phytosterols, esterified phytosterol, non esterified phytosterol, zeaxanthine, caffeine, and a combination thereof.

28. Method according to claim 2, wherein the LPA is selected from the group consisting of long-chain alcohols, fatty acids, pegylated fatty acids, glycerol fatty acid esters, monoglycerides, diglycerides, derivatives of mono-diglycerides, pegylated vegetable oils, sorbitan esters, poloxyethylene sorbitan esters, propylene glycol mono- or diesters, phospholipids, phosphatides, cerebrosides, gangliosides, cephalins, lipids, glycolipids, sulfatides, sugar esters, sugar ethers, sucrose esters, sterols, and polyglycerol esters.

29. Method according to claim 2, wherein the LPA is selected from the group consisting of myristic acid, oleic acid, lauric acid, stearic acid, palmitic acid, PEG 1-4 stearate, PEG 2-4 oleate, PEG-4 dilaurate, PEG-4 dioleate, PEG-4 distearate, PEG-6 dioleate, PEG-6 distearate, PEG-8 dioleate, PEG-3-16 castor oil, PEG 5-10 hydrogenated castor oil, PEG 6-20 corn oil, PEG 6-20 almond oil, PEG 6 olive oil, PEG-6 peanut oil, PEG-6 palm kernel oil, PEG-6 hydrogenated palm kernel oil, PEG-4 capric/caprylic triglyceride, mono, di, tri, tetraesters of vegetable oil and sorbitol, pentaerythrityl di, tetra stearate, isostearate, oleate, caprylate or caprate, polyglyceryl-e dioleate, stearate, or isostearate, plyglyceryl 4-10 pentaoleate, polyglyceryl 2-4 oleate, stearate, or isostearate, polyglyceryl 4-10 pentaoleate, polyglyceryl-3 dioleate, polyglyceryl-6 dioleate, polyclyceryl-10 trioleate, polyglyceryl-3 distearate propylene glycol mono- or diesters of C₆ to C₂₀ fatty acid, monoglycerides of C₆ to C₂₀ fatty acid, lactic acid derivatives of monoglycerides, lactic acid derivatives of diglycerides, diacetyl tartaric ester of monoglycerides, triglycerol monostrearate cholesterol, phytosterol, PEG 5-10 soya sterol, PEG-6 sorbitan tetra, hexasteararate, PEG-6 sorbitan tetraoleate, sorbitan monolaurate, sorbitan monopalmitate, sorbitan mono trioleate, sorbitan mono and tristearate, sorbitan monoisostearate, sorbitan sesquioleate, sorbitan sesquistearate, PEG-2-5 oleyl ether, POE 2-4 lauryl ether, PEG-2 cetyl ether, PEG-2 stearyl ether, sucrose distearate, sucrose dipalmitate, ethyl oleate, isopropyl myristate, isopropyl palmitate, ethyl linoleate, isopropyl linoleate, poloxamers, phospholipids, lecithins, cephalins, oat lipids and lipophilic amphiphilic lipids from other plants; and mixtures thereof.

30. Method according to claim 2, wherein the emulsifier is selected from the group consisting of low molecular weight surfactants having a HLB>8, proteins from milk, proteins from soya, amino acids peptides, protein hydrolysates, block co-polymer, random co-polymers, Gemini surfactants, surface active hydrocolloids, apoprotein-like biopolymers, polyelectrolyte-surfactant complexes, DNA, nucleic acid, particles (micro or nano-sized), starch and starch-based polymers, amylase, amylopectin and mixtures thereof.

31. Method according to claim 2 for the delayed release of active elements during a state selected from the group consisting of storage, consumption and digestion.

32. Method according to claim 2 for the delayed release in the mouth.

33. Method according to claim 3 which is used for the delayed release of active elements such that the release of at least one active element, which has a octanol/water partitioning coefficient logP greater than 0, corresponds to a greater Tmax than the Tmax obtained for a standard oil-in-water emulsion where no lipophilic additive is used.

34. Method according to claim 3 wherein the Tmax is increased by a factor greater than 1.15 compared to the Tmax measured from a standard oil-in-water emulsion containing the same oil content but where no lipophilic additive is used.

35. Method according to claim 3 wherein the oil droplets have a diameter of 5 nm to hundreds of micrometers and the oil-in-water emulsion contains the active element which is present at between 0.0001 part per million (ppm) and 80% based on the total composition.

36. Method according to claim 3 comprising dispersed oil droplets having interfaces, between lipophilic domains and hydrophilic or amphiphilic domains, created by the lipophilic additives and comprising:
an oil selected from the group consisting of mineral oils, hydrocarbons, vegetable oils, waxes, alcohols, fatty acids, mono-, di-, tri-acylglycerols, essential oils, flavouring oils, lipophilic vitamins, esters, neutraceuticals, terpins, terpenes and mixtures thereof;
a lipophilic additive (LPA) or mixtures of lipophilic and hydrophilic additives, having a resulting HLB value (Hydrophilic-Lipophilic Balance) lower than about 10;
hydrophilic or amphiphilic domains in the form of droplets or channels comprising water or a non-aqueous polar liquid, such as a polyol;
and
an aqueous continuous phase, which contains a hydrophilic emulsifier.

37. Method according to claim 3, wherein the active element is selected from the group consisting of flavours, flavour precursors, aromas, aroma precursors, taste enhancers, salts, sugars, amino-acids, polysaccharides, enzymes, peptides, proteins or carbohydrates, food supplements, food additives, hormones, bacteria, plant extracts, medicaments, drugs, nutrients, chemicals for agro-chemical or cosmetical applications, carotenoids, vitamins, antioxidants or nutraceuticals selected from the group comprising of lutein, lutein esters, β-carotene, tocopherol, tocopherol acetate, tocotrienol, lycopene, Co-Q₁₀, flax seed oil, fish oil, omega-e oils, omega-6 oils, DHA, EPA, arachidonic-rich oils, LCPUFA oils, menthol, mint oil, lipoic acid, vitamins, polyphenols and their glycosides, ester and/or sulphate conjugates, isoflavones, flavonols, flavanones and their glycosides, flavan 3-ols comprising catechin monomers and their gallate esters, vitamin C, vitamin C palmitate, vitamin A, vitamin B₁₂, vitamin D, α- and γ-polyunsaturated fatty acids, phytosterols, esterified phytosterol, non esterified phytosterol, zeaxanthin, caffeine, and a combination thereof.

38. Method according to claim 3, wherein the LPA is selected from the group consisting of long-chain alcohols, fatty acids, pegylated fatty acids, glycerol fatty acid esters, monoglycerides, diglycerides, derivatives of mono-diglycerides, pegylated vegetable oils, sorbitan esters, poloxyethylene sorbitan esters, propylene glycol mono- or diesters, phospholipids, phosphatides, cerebrosides, gangliosides, cephalins, lipids, glycolipids, sulfatides, sugar esters, sugar ethers, sucrose esters, sterols, and polyglycerol esters.

39. Method according to claim 3, wherein the LPA is selected from the group consisting of myristic acid, oleic acid, lauric acid, stearic acid, palmitic acid, PEG 1-4 stearate, PEG 2-4 oleate, PEG-4 dilaurate, PEG-4 dioleate, PEG-4 distearate, PEG-6 dioleate, PEG-6 distearate, PEG-8 dioleate, PEG-3-16 castor oil, PEG 5-10 hydrogenated castor oil, PEG 6-20 corn oil, PEG 6-20 almond oil, PEG 6 olive oil, PEG-6 peanut oil, PEG-6 palm kernel oil, PEG-6 hydrogenated palm kernel oil, PEG-4 capric/caprylic triglyceride, mono, di, tri, tetraesters of vegetable oil and sorbitol, pentaerythrityl di, tetra stearate, isostearate, oleate, caprylate or caprate, polyglyceryl-e dioleate, stearate, or isostearate, plyglyceryl 4-10 pentaoleate, polyglyceryl 2-4 oleate, stearate, or isostearate, polyglyceryl 4-10 pentaoleate, polyglyceryl-3 dioleate, polyglyceryl-6 dioleate, polyclyceryl-10 trioleate, polyglyceryl-3 distearate propylene glycol mono- or diesters of C₆ to C₂₀ fatty acid, monoglycerides of C₆ to C₂₀ fatty acid, lactic acid derivatives of monoglycerides, lactic acid derivatives of diglycerides, diacetyl tartaric ester of monoglycerides, triglycerol monostrearate cholesterol, phytosterol, PEG 5-10 soya sterol, PEG-6 sorbitan tetra, hexasteararate, PEG-6 sorbitan tetraoleate, sorbitan monolaurate, sorbitan monopalmitate, sorbitan mono trioleate, sorbitan mono and tristearate, sorbitan monoisostearate, sorbitan sesquioleate, sorbitan sesquistearate, PEG-2-5 oleyl ether, POE 2-4 lauryl ether, PEG-2 cetyl ether, PEG-2 stearyl ether, sucrose distearate, sucrose dipalmitate, ethyl oleate, isopropyl myristate, isopropyl palmitate, ethyl linoleate, isopropyl linoleate, poloxamers, phospholipids, lecithins, cephalins, oat lipids and lipophilic amphiphilic lipids from other plants; and mixtures thereof.

40. Method according to claim 3, wherein the emulsifier is selected from the group consisting of low molecular weight surfactants having a HLB>8, proteins from milk, proteins from soya, amino acids peptides, protein hydrolysates, block co-polymer, random co-polymers, Gemini surfactants, surface active hydrocolloids, apoprotein-like biopolymers, polyelectrolyte-surfactant complexes, DNA, nucleic acid, particles (micro or nano-sized), starch and starch-based polymers, amylase, amylopectin and mixtures thereof.

41. Method according to claim 3 for the delayed release of active elements during a state selected from the group consisting of storage, consumption and digestion.

42. Method according to claim 3 for the delayed release in the mouth.

43. Method according to claim 11, wherein the emulsifier is selected from the group consisting of whey proteins, whey protein isolates, whey protein concentrates, whey protein aggregates, caseinates, casein micelles, caseins, lysozyme, albumins, gum Arabic, xanthan gum, gelatine, polyelectrolytes, carrageenans, carboxymethylcellulose, cellulose derivatives, Acacia gum, galactomannans, chitosans, hyaluronic acid, pectins, propylene glycol alginate, modified starches, Portulaca Oleracean, Tragacanth, gellan gum, protein-polysaccharide, protein-protein, or polysaccharide polysaccharide hybrids, conjugates, or mixtures of polymers and biopolymers.

44. Method according to claim 30, wherein the emulsifier is selected from the group consisting of whey proteins, whey protein isolates, whey protein concentrates, whey protein aggregates, caseinates, casein micelles, caseins, lysozyme, albumins, gum Arabic, xanthan gum, gelatine, polyelectrolytes, carrageenans, carboxymethylcellulose, cellulose derivatives, Acacia gum, galactomannans, chitosans, hyaluronic acid, pectins, propylene glycol alginate, modified starches, Portulaca Oleracean, Tragacanth, gellan gum, protein-polysaccharide, protein-protein, or polysaccharide polysaccharide hybrids, conjugates, or mixtures of polymers and biopolymers.

45. Method according to claim 40, wherein the emulsifier is selected from the group consisting of whey proteins, whey protein isolates, whey protein concentrates, whey protein aggregates, caseinates, casein micelles, caseins, lysozyme, albumins, gum Arabic, xanthan gum, gelatine, polyelectrolytes, carrageenans, carboxymethylcellulose, cellulose derivatives, Acacia gum, galactomannans, chitosans, hyaluronic acid, pectins, propylene glycol alginate, modified starches, Portulaca Oleracean, Tragacanth, gellan gum, protein-polysaccharide, protein-protein, or polysaccharide polysaccharide hybrids, conjugates, or mixtures of polymers and biopolymers.
